(19) United States
(12) Patent Application Publication
(10) Pub. No.: US 2017/0303487 A1
(43) Pub. Date: Oct. 26, 2017

(54) METHOD FOR SELECTING ELITE TREES OF PONGAMIA PINNATA

(71) Applicant: Peterson Technologies, LLC, Canton, MI (US)

(72) Inventor: M.V. R. PRASAD, Banjara Hills (IN)

(21) Appl. No.: 15/643,730

(22) Filed: Jul. 7, 2017

Related U.S. Application Data
(63) Continuation of application No. PCT/US2016/012469, filed on Jan. 7, 2016.
(60) Provisional application No. 62/100,552, filed on Jan. 7, 2015.

(51) Int. Cl. A01H 1/04

(52) U.S. Cl. CPC A01H 1/04

(57) ABSTRACT

A method for selecting genetically elite trees of Pongamia pinnata is disclosed. This method relates to the early measurement of tree characteristics for selecting Pongamia trees having higher than average productivity levels, based on the selection of characteristics that are stable in expression and having close linkage with kernel yield. This invention relates to tree productivity. More particularly, this invention relates to a method for the selection, propagation, and plantation planting of genetically elite trees of Pongamia pinnata.

### RELATED APPLICATIONS

This application is a continuation of PCT application No. PCT/US2016/012469, filed Jan. 7, 2016, which claims the benefit of priority of United States provisional application No. 62/100,552, filed Jan. 7, 2015, the disclosures of which are hereby incorporated by reference as if written herein in their entirety.

### FIELD OF THE INVENTION

This disclosure relates to a method for selecting Pongamia trees for yield improvement of favorable traits, notably kernel/seed yield. More particularly, this disclosure relates to the propagation and system planting of genetically elite trees of Pongamia pinnata.

### BACKGROUND OF THE INVENTION

Pongamia pinnata (also known as Millettia pinnata) is a fast growing evergreen deciduous tree that is an Indo-Malaysian species common in alluvial and coastal environments from India to Fiji including northern Australia, New Guinea, Malaysia, Southern China, Vietnam, and Indonesia. Pongamia pinnata is a “tree legume” in that it comprises Rhizobium-nodulated roots that enable symbiotic nitrogen fixation. It also can use mineralized nitrogen in the form of nitrate. Traditionally, Pongamia pinnata has been cultivated for ornamental gardens because of its attractive and abundant Wisteria-like flowers and abundant green foliage, and also for a variety of practical uses such as making cooking stove fuel, compost, strings and ropes and for extracting a black gum from its bark that is used to treat wounds caused by poisonous fish and in other traditional remedies. The seeds contain an oil (about 25-40% by weight) known as “karanj” or “pongam”or “honge” oil, which is a bitter, red brown, thick, non-drying, non-edible oil, which is used for tanning leather, in soap, as a liniment to treat scabies, herpes, and rheumatism and as an illuminating oil. This seed oil has a high content of triglycerides (containing up to about 55% oleic acid) which, in combination with the hardiness of the tree in poor soil conditions, has made Pongamia pinnata an attractive source of oil for the production of biofuels.

### SUMMARY OF THE INVENTION

The present disclosure is a method for selecting genetically elite trees of Pongamia pinnata. This method relates to the early measurement of tree characteristics for selecting Pongamia trees having higher than average productivity levels, based on the selection of characteristics that are stable in expression and having close linkage with kernel/seed yield. This invention relates to tree productivity. More particularly, this invention relates to a method for the selection, propagation, and plantation planting of genetically elite trees of Pongamia pinnata.

In one embodiment, the disclosure provides a method for selecting an elite Pongamia tree for breeding from a group of Pongamia trees, based on favorable traits, related to kernel/seed yield.

In a second embodiment, these favorable and measurable traits are chosen from the group comprising globose canopy, extended canopy area, shelling percentage, and mass of total kernels/seeds per Pongamia tree.

In yet another particular embodiment, an elite Pongamia tree is selected for breeding if it has a globose canopy has a radius of at least 3.0 m, an extended canopy area is at least 12 square meters, a shelling percentage at least 40% greater than the shelling percentage of a non-elite Pongamia pinnata tree, and a kernel/seed yield at least 50 kg greater than a kernel/seed yield of a non-elite Pongamia pinnata tree.

In another embodiment, the disclosure provides a method for the propagation of genetically elite Pongamia pinnata trees which is not carried out through the use of seed.

With this in mind, there is a need in the art to identify stable, phenotypic characteristics associated with higher than average productivity (i.e., kernel/seed yield) which result in the selection of genetically elite Pongamia pinnata trees.

### DETAILED DESCRIPTION OF THE INVENTION

The definitions and methods provided define the present disclosure and guide those of ordinary skill in the art in the practice of the present disclosure. Unless otherwise noted, terms are to be understood according to conventional usage by those of ordinary sill in the relevant art.

The present disclosure relates to a method for selecting elite trees of Pongamia pinnata. This species common in alluvial and coastal environments from India to Fiji including northern Australia, New Guinea, Malaysia, Southern China, Vietnam, and Indonesia.

The method of the present disclosure may be used in selecting trees based upon characteristics measured in those trees using the methods of the present disclosure. Several studies carried out on tree productivity/yield have indicated that kernel/seed yield is more reliable parameter than the pod yield, particularly in examples where the kernel/seed yield is a product of economic value (Salam et al. 2010). However, selection for kernel/seed yield in a mature tree is not apparently feasible, without undertaking the steps of harvesting pods and shelling the harvested pods, which are not easy in tree breeding programs. Selection for pod yield alone, based on the visual observation of the tree and further harvesting, was not found to be reliable as pod yield is highly variable and subjected to seasonal influences. Therefore, the selection of a genetically superior Pongamia pinnata tree for consistently higher productivity/yield cannot be made based on pod yield alone.

The kernel/seed yield of a Pongamia pinnata tree depends upon the canopy growth which again is a function of the canopy components such as the shape, the kind of canopy, its branching, and reproductive efficiency. Hence, canopy dry matter, its spread, and conversion into reproductive tissues have a direct bearing on tree productivity/yield. One way of assessing the consistent reproductive efficiency of a tree is to arrive at a reliable estimate of the relationship of its canopy with seed/kernel yield. Such a relationship could be translated into dependable selection criteria in tree breeding for seed yield. One such exercise could be to arrive at a reliable estimate of canopy dry-matter and its harvest index. The Pongamia pinnata trees combining high canopy dry matter and high harvest index could be high yielding trees (Kumakov et al. 2001, Soltani 2006, Marcelis 1994, 1996).

In another embodiment the method of the present disclosure may be used in selecting trees based upon canopy attributes and their relationship/association with seed/kernel yield. The canopy characters that are stable in their expression and show strong linkage or association with seed/kernel yield can establish reliable criteria for selecting genetically superior and consistently high yielding elite Pongamia pinnata trees to enhance the efficiency of tree breeding programs for kernel/seed yield (Prasad et al. 2000, Aliyu 2006, Samal et al. 2003).

In another embodiment, the concept of growing Pongamia in an organized plantation has not taken roots in India. Hence there are differences between Pongamia planted for afforestation and Pongamia plantations as described herein have to be understood and appreciated. In the case of Pongamia used for afforestation, the tree becomes a small component of the complex tree populations used for this activity. Pongamia trees planted in these afforestation program cannot be managed towards achieving sustainable levels of economic productivity.

In the case of Pongamia plantations, high quality, genetically elite grafts can be obtained from planting material of only one species of Pongamia pinnata. The Pongamia plantation is raised in a definite plantation geometry, and population density is managed scientifically to achieve economically sustainable levels of productivity.

As used herein, the term “elite” means a genotype that has a culmination of many distinguishable traits such as seed/kernel yield, pod yield, vegetative vigor, and disease resistance, a breeder to harvest a product of commercial significance.

As used herein, the “globose canopy” is the distance from the trunk of a Pongamia tree to the edge of the canopy.

As used herein, “shelling percentage” can be used to convert unshelled harvest weights to estimates of shelled weights. To get an estimate of yield when a producer has not yet shelled his harvest: weigh the unshelled harvest, weigh a small shelling percentage sub-sample (0.5 kilograms), shell, and then reweigh the shelled sub-sample.

As used herein, “elite” can refer to Pongamia trees or Pongamia plant material, i.e., root-stocks and scions that is adapted (selectively bred and optimized to new surroundings (i.e., environment).

Conversely, as used herein, the term “non-elite” can refer to a Pongamia tree that does not possess favorable agronomic traits, notably kernel yield, which are of commercial significance.

As used herein, the term “comprising” means “including but not limited to”.

### EXAMPLE 1

### Selecting Elite Trees of Pongamia pinnata

The methods disclosed herein describe the various embodiments for the selection of elite trees of Pongamia pinnata for consistent higher productivity levels. The methods relate to the measurement of Pongamia pinnata tree characteristics based on Pongamia pinnata tree characteristics which are stable in their expression and have close linkage to kernel/seed yield. As described herein, the selection is not based on total kernel/seed yield alone, as kernel/seed yield can vary from year to year. The phenotypic characteristics are described in Table 1 with measurements.

TABLE 1

| Phenotypic Characteristic | Measurement Index |
|---|---|
| Globose canopy | With radius of +3.0 m, i.e., distance |
| | from the trunk to the edge of the canopy. |
| Extended canopy area | +12 m² |
| A higher shelling percentage | +40% |
| Higher kernel/seed yield per | +50 kg |
| tree | |

The kernel/seed yield is estimated from a pod yield harvested from a square meter area of canopy and multiplied by shelling percentage calculated from a representative sample of shelled pods.

### EXAMPLE 2

### Elite Tree Replication of Pongamia pinnata

The propagation of the elite trees is not carried out through seeds. The scions procured from the elite trees are grafted on to 60 to 120 days of young root-stocks raised from bold and well developed seeds adopting a cleft-wedge grafting.

Root-stocks. Bold and well developed seeds each weighing about 1.5g are selected and pre-treated in hot water of 60° C. for 25-30 minutes and planted in poly-bags filled with and mixture of sand and farmyard manure (FYM) (3:1:0.5) and watered daily. The germination occurs in about two weeks. At 60 days following germination less vigorous and stunted seedlings are culled out and rejected. Robust and disease-free seedlings representing well developed root-systems are selected as root-stocks for grafting. The selected vigorous root stocks should have at least 4 pairs of leaves and height of 10″ to 12″ (around 0.3 m).

Scions selection. Scions are the mature branches with dormant terminal and axillary buds showing no fresh growth that are obtained from the selected Pongamia elite tree to be propagated vegetatively. The color of the scion tissues should be greyish brown to dark brown and must be free from any disease or pest attack. The length of the scion should be +6″ and the girth at the bottom of the scion stick should be of a thickness of a pencil. This means that scions should have enough reserves and must be of the age of over 90 days. The scions are normally procured from the elite trees following a pre-curing of the branches selected for scions by cutting off the leaf laminae (leaving petioles intact) before their separation from the elite mother tree.

Grafting Process. The selected root-stock is defoliated leaving two pairs of leaves at the bottom. The terminal bud of the root-stock is cut-off and a longitudinal cut all along 6 to 8 cm length from the decapitated top of root-stock is made with clean and sterilized sharp grfating knife. A scion is cut tangentially at the base and prepared in the form a wedge pointing to the base, matching with the cleft made on the root-stock. The wedge of the scion is inserted into the cleft of the root-stock taking care to match the cambial layers of the root-stock and scion. The cleft of the root-stock is closed and bound with the wedge of the scion forming a firm union and fastened with a grafting tape securely. A long and narrow polyethylene cover is used to cover the grafted scion portion taking care to ensure that tip of the cover does not come into contact with the scion.

After care of the grafts. The grafts are kept under total shade ensuring a humidity of over 55% and watered every day. The polythene caps over the grafts are removed after a week or ten days following grafting. The dormant buds of the scion start sprouting after two weeks following grafting. Any sprouts below the graft union that represent root-stock tissues are nipped off and not encouraged to sprout. The developing grafts are retained under shade and watered regularly till the emerging scion leaves turn dark green, when the bottom two pairs of root-stock leaves are removed. The grafting tape around the graft-union is removed after 90 days following grafting when the graft would be ready for transplantation in the main field.

### EXAMPLE 3

### Plantations of Elite Pongamia pinnata Trees

The method for developing plantations for elite Pongamia pinnata trees is described herein. The methodology discloses a procedure for planting, care of saplings, tree training, nutrient management, and control of pests and diseases to ensure optimum growth of the trees in a range of climatic and soil conditions. The selection of proper location and site, planting system and planting distance, and choosing the planting materials are carefully considered to ensure maximum production.

Certain plantation management strategies specific to Pongamia are as follows:
1. Maintain diversity of genotypes: Grow clones from diverse elite trees rather than those from one or two mother trees, if there is no consideration of a specific product quality from plantation.
2. Eco-friendly management: Avoid use of chemical pesticides and other strong chemicals that would harm the symbiotic biological systems.
3. Ensure soil conservation avoiding erosion and carry out water harvesting by collecting run-off in farm ponds exploiting the natural slope.
4. Nutritional management must be effected through mostly organic sources. Avoid giving chemical N fertilizers.
5. Plantation management methods include mulch of grass, drip irrigation using rain water collected from run-off and above all creating a tree canopy architecture conducive for productivity through the adoption of the pruning techniques in relation to light interception, CO₂ assimilation, carbohydrate partition, stomatal resistance, water potential and so on.
6. To them must be added energy inputs, efficient use of natural resources (H₂O, light, CO₂, nutrients), environmental impact, and changes in the agri-ecosystem, all the while bearing in mind that the grower must produce and be competitive in a market

Planting. Pongamia bio-energy plantations may be planted in the pre-dug pits (size: 45 to 50 cubic cm) spaced at 4×4 m. The top soil dug out from each pit may be mixed with 4 kg of well decomposed FYM or 2 kg of Poultry manure +200 g of Single Super Phosphate +40 g of Micronutrient Mixture per pit and the pit re-filled before planting. The above recommendations are applicable for normal problem less soils. In the case of saline/alkaline soils and soils with specific toxic pollutants etc., the recommendations with regard to the nutrients/soil amendments to be applied should be arrived at based on soil analysis data.

Planting on Rocky Terrains. In areas with exposed rock, artificial explosion can be used for pit preparation for planting high value Pongamia trees. Powder is used to explode a pit to a depth of 1 m and a diameter of 2-3 m. Then good top soil and organic matter are put into the pit into which a graft is planted and watered. The nitrogen from the explosion powder remains in the pit after explosion thus increasing the nutrients for tree growth.

The following two important steps should be implemented to ensure proper seedling establishment and zero plant mortality in the first year of the plantation.
1. Undertake planting of Pongamia trees only when the soil profile is saturated with rain water to a depth of more than 60 cm.
2. Mix the dugout soil with 100 g of neem cake or 4 to 5 g of carbofuron and fill the pit at planting to ward off damage to root by any soil born biotic agents.

These above recommendations are tentatively proposed for normal soil.

The nutrient schedule recommended should be based on the soil test values. It is recommended that the soil of the site marked for plantation is analysed for basic nutrient parameters listed below:
(i). Soil pH
(ii). Soil organic carbon content (%)
(iii) Soil organic matter content (%)
(iv). Total hydrocarbon content (%)
(v). Ece (ds/m)
(vi). Soil bulk density (kg/m3)
(vii). Exchangeable Sodium (Na) (%)
(viii). Cation Exchange Capacity (meq/100 g of soil) and
(ix). Calcium carbonate content (%)

Care of Saplings. In order to keep the grafted sapling stable, the soil around it should be pressed by hand and the base of the sapling covered with grass straw after planting. It would be advisable to provide support of a strong stick fixed adjoining the planted sapling to which the sapling could be fastened, so as to ensure that the sapling does not lodge and would grow erect without any sort of crookedness of the main stem. Form a basin around the sapling with bund erected around it in semi-circular fashion leaving the opening to collect run-off exploiting the natural slope of the terrain.

Carry out periodical inter-cultures to achieve efficient weed control. During the first summer/rainless hot period following planting, watering of saplings with 30 to 40 litres of water/plant either from water harvested from run-off or other source would ensure proper canopy development.

Tree Training and Pruning. The pruning recommended in respect of root-stock sprouts and extensive branches must be carried out totally as the tree grows. Remove all sprouts and shoot growth below the graft joint. Remove the lower branches with secateurs so that the plant has a free trunk up to a height of one meter by the 4th and 5th year of planting. Carry out this operation each year. This facilitates better culture and avoids the stem-borer infestation. Remove the flowering panicles in the first two years to encourage good branching. Allow the first fruiting in the third to the fourth year after planting. Behead the main branch growing vertically after 3^{rd} year harvest at a height of 2.5 to 3.0 meters. This stimulates good canopy growth conducive for higher yields. Beware of root suckers, which are produced abundantly by the tree's surface root system. These suckers that appear periodically represent root-stock tissue and as such should be eliminated immediately after their appearance without any delay. Any failure to attend to this important job would result in stunting of scion canopy leading to poor yields. If the suckers are allowed to grow, quality of the produce and total yield will be drastically altered to negative side in the succeeding seasons/years. Protect the main trunk by smearing Bordeaux paste or bitumen. This will avoid damage to the trunk by pests and diseases. Pruning in the early years after planting is aimed at shaping the tree to give it a balanced structure and to prevent the growth of multiple main branches at the same level. Particular attention is paid to the formation of a single trunk in the first year. Green pruning is performed in the second year to select the future main branches, usually three or four well positioned branches around the trunk. Subsequently pruning is performed each year after harvest to remove the following:
1. Curved branches and those that have given pods in the preceding cycles
2. Withered shoots and diseased branches.
3. Badly positioned shoots including the small erect shoots that occur on productive intensive branches or an over-abundance in order to make openings in the foliage. In order to prevent the spread of diseases, pruning tools should be disinfected with concentrated bleach and large pruning wounds protected by wound dressing through the application of Bordeaux Paste.
4. A major component of plantation management is corrective pruning to enhance the canopy efficiency to reap higher productivity levels.
5. In the reproductive phase, retain intensive branches and selectively eliminate the extensive branches.
6. Intensive branches are the flowering branches which continuously generate lateral reproductive branches. They have high frequency of reproductive nodes.
7. Extensive branches are totally vegetative branches devoid of any reproductive tissues. Extensive branches keep on generating vegetative branches without any flowering. Sometimes few flowers may be seen in the lateral extensive branches without any conspicuous yield advantage.

Nutrient care. As flowering season approaches in August, September, and October, trees need to be replenished with nutrients in order to have enough food for the developing pods. The decision for supplementary nutrition may be taken depending upon the growth and development of canopy. If the growth of sapling is stunted after the first year, the following procedure may be followed. For this a small trench of 30 cm deep, 120 to 150 cm long and 40 to 60 cm wide should be made around the tree canopy. Around 2 kg of FYM +50 g of single superphosphate (SSP) may be applied in the trench dug around the plant and the trench closed with the dugout top soil. If the symptoms of micronutrient deficiency are observed, the following corrective measures must be taken. Annual dressing is aimed mainly at conserving the soil fertility level by compensating the exploited minerals calculated from harvest data. Soil and leaf analyses are frequently used complementary tools for decision-making. Micronutrient trace elements such as magnesium, iron, zinc, manganese, copper, boron, molybdenum, etc. are rarely applied systematically. These elements are sprayed on to fully developed young leaves of the spring growth, if leaf analysis and symptoms show the need.

Magnesium. Signs of this deficiency are found only in adult leaves. Discoloration starts in the median part of the lamina and spreads gradually to the base and tip of the leaf in a V-shaped pattern. In the final stage, only a V-shaped part close to the petiole is not discoloured. The young leaves of trees suffering from this deficiency are never affected. Spray new leaves with 1.2% magnesium nitrate on completion of lamina elongation.

Iron. The deficiency is shown by overall discoloration of the lamina, which turns pale green to yellow. In contrast with nitrogen deficiency, the vein pattern remains green. True soil iron deficiency is rare. It is more frequently a blockage of assimilable forms of iron. If possible, first correct the physical causes of the deficiency: hydromorphy, alkaline pH, etc. Apply chelated forms of iron to the soil.

Zinc. A frequent deficiency in all areas. It is seen in the young leaves which remain small and erect. The lamina is seriously discolored, from pale green to yellow, in all the inter vein parts. The veins and halo along each of them remain green. Spraying new leaves with zinc sulphate (2.5 g/l) on completion of lamina elongation.

Manganese. This is another common deficiency. It is similar to zinc deficiency but differs in two respects: the leaves are not deformed and remain their normal size and lamina discoloration is less intense. Spraying new leaves completing lamina elongation with manganese sulphate (1.5 g/l).

Boron. This fairly rare deficiency is shown by the suberisation of the veins on the underside of the lamina. The young shoots may wither. The distance between deficiency and toxicity is small and so boron applications must be carefully measured. Spraying with 2.5 g/l Solubor.

Despite lack of any clear data regarding the yield losses in Pongamia due to micronutrient deficiencies, symptoms of deficiencies of certain micronutrients, notably that of Zinc and Magnesium have been observed by the author at different stages of plant growth, particularly when no organic matter was applied at planting. Application of organic matter in the form of well decomposed FYM at 4 kg/plant or 2 kg of poultry manure/plant or 1.5 kg of Organic Matter (Annapurna of Multiplex) would be necessary to ward of any micronutrient deficiencies. The necessary management practices in this regard are already listed in the preceding paragraphs.

Pest and Diseases. Although Pongamia pinnata attracts many pests and diseases, it is unique in the sense that the biotic stresses have not caused much of yield reduction. Some of the important pests are Parnara mathias, Gracillaria spp, Indarbela quadrinotata, Myllocerus curvicornis, and Acrocercops spp. Attacks by these insects cause whitish streaks and the formation of galls on affected leaves. Several fungi attack the seedlings and the trees. Ganoderma lucidum causes root rot and Fomes merilli (Murrill) Sacc. & Trott attack the tender shoots and leaves and cause early defoliation in the seedlings and trees. The eriophyid mite (Eriophyes cherianei and/or Aceria pongamiae) causes leaf galls; but no yield reduction was observed. However, in susceptible trees, apart from leaf galls, the pods are transformed into small globose sterile structures due to the attack of midge (Asphondylia pongamiae). Care should be taken to avoid selecting such trees for propagation. Incidence of pod borer has been noticed; but the attack of pod borer was found in the case of ill filled pods and the pods that develop in the initial fruiting season. Synchronous pod bearing trees did not exhibit much of pod borer incidence. Pongamia leaf miner has not been found to cause any yield reduction. Phytophthora gummosis is certainly the world's most widespread fungal disease. Three Phytophthora species are involved in particular: P. parasitica, P. citrophthora and P. palmivora. The pathogenic fungus is endemic in the soil in all zones. It is particularly active during hot, humid weather. Phytophthora can survive in a suspended form during dry periods that are unfavorable for its development. In citrus, attacks may occur at all stages of cultivation on both young seedlings and adult trees in orchards. In the parts of the plants attacked, the fungus is found in living tissue close to the lesions. The attacks destroy the bark on trunks and low branches; this strongly disturbs the translocation of elaborate sap and causes flows of gum and the yellowing of foliage upstream of the area of bark that has been destroyed. Other less visible symptoms include root rot. Unless trunk and root attacks are controlled at an early stage they will cause the total or partial withering of the tree. In the case of Pongamia, however, incidence of this disease is not very common.

What is claimed is:

1. A method for selecting an elite Pongamia tree for breeding from a group of Pongamia trees, comprising:
a. measuring the globose canopy
b. measuring the extended canopy area
c. measuring shelling percentage
d. weighing total kernels/seeds per Pongamia tree ; and
e. selecting an elite Pongamia tree for breeding if it has
i. a globose canopy has a radius of at least 3.0 m;
ii. an extended canopy area is at least 12 square meters;
iii. a shelling percentage at least 40% greater than the shelling percentage of a non-elite Pongamia pinnata tree; and
iv. a kernel/seed yield at least 50 kg greater than a kernel/seed yield of a non-elite Pongamia pinnata tree.

2. A propagated elite Pongamia tree comprising a root-stock and scion wherein the scion is procured from an elite Pongamia tree of claim 1.

3. A propagated elite Pongamia tree of claim 2 wherein the scion is grafted on to a root-stock greater than 60 days old but less than 120 days old.

4. A propagated elite Pongamia tree of claim 2 wherein the root-stock has at least four pairs of leaves.

5. A propagated elite Pongamia tree of claim 2 wherein the root-stock has a height of at least 0.3 meters.
